(19) United States
(12) Patent Application Publication
(10) Pub. No.: US 2013/0101527 A1
(43) Pub. Date: Apr. 25, 2013

(54) METHODS, COMPOUNDS AND PHARMACEUTICAL COMPOSITIONS FOR TREATING NEUROLOGICAL DISORDERS

(71) Applicants: Rodolfo R. Llinas, New York, NY (US); Emeline L. Maillet, Bagneux (FR)

(72) Inventors: Rodolfo R. Llinas, New York, NY (US); Emeline L. Maillet, Bagneux (FR)

(21) Appl. No.: 13/651,459

(22) Filed: Oct. 14, 2012

Related U.S. Application Data
(60) Provisional application No. 61/547,676, filed on Oct. 14, 2011.

(51) Int. Cl. A61K 47/26; A61K 31/045

(52) U.S. Cl. CPC A61K 47/26; A61K 31/045
USPC 424/48; 514/724

(57) ABSTRACT

Pharmaceutical compositions or formulations containing octanol are provided. The pharmaceutical compositions are useful for the prevention and treatment of a variety of conditions in mammals including humans, including neurological disorders such as thalamocortical dysrhythmia disorder.

### RELATED APPLICATION

The present application claims the benefit under 35 U.S.C. §119 of U.S. Provisional Application Ser. No. 61/547,676 filed Oct. 14, 2011. The contents of said provisional application is hereby incorporated by reference in its entirety.

### FIELD OF THE INVENTION

This invention relates to novel compositions, pharmaceutical compositions and formulations comprising a C₂-C₁₀ alkanol useful in treating neurological disorders. Specifically, the invention relates to novel pharmaceutical compositions comprising octanol and cyclodextrin. This invention also relates to methods for the prevention, prophylaxis and/or treatment of conditions that are causally related to thalamocortical dysrhythmia. The invention further relates to novel formulations of octanol encapsulated in cyclodextrin. The invention also relates to novel low dosage formulations of octanol. The invention also further relates to novel low dosage octanol formulations incorporated in mouth meltable pills, mouth meltable strips, sublingual micro pills, gum, candy, ointment, gel, systemic lotion, systemic gel, massage cream, massage gel, mouth meltable pills, capsules, homeopathic pills, hair lotion, or mouth wash.

### BACKGROUND OF THE INVENTION

Neurological disorders that affect the central nervous system, the peripheral nervous system, and the autonomic nervous system strike millions of people worldwide.

These varied disorders include, but are not limited to, thalamocortical dysrhythmia, neurogenic pain, obsessive-compulsive disorder, depression, panic disorder, Parkinson's disease, schizophrenia, rigidity, dystonia, tinnitus, tremor, and epilepsy. In particular, these and other neurological disorders occur when the coordinate, controlled electrical activity at the cortical level of the brain becomes disrupted, thereby leading to uncoordinated electrical activity and abnormal neuronal oscillation.

In particular, thalamocortical dysrhythmia refers to a neurological and/or psychiatric condition arising from the abnormal rhythmicity in particular components of the thalamocortical circuit (Llinas et al. (1999) Proc. Natl. Acad. Sci. USA, 96:15222-15227). At the cellular level, the abnormal activity of the thalamic neurons is caused by an increase of low frequency oscillatory activity due to protracted activation of the T type calcium channels because of direct modification of the channel properties or more commonly, abnormal hyperpolarization of the thalamic neurons due to excess inhibition. Such abnormal activity is transmitted to the related cortical area to which the given thalamic neurons are oscillating generating a recurrent attractor that is maintained by the recursive nature of the circuit. At the macrocellular level, the abnormal rhythmicity interferes with the communication among and between different regions of the brain, and thereby impairs the motor and cognitive skills that are controlled by those regions of the cortex.

Spike output in neuronal cell types is affected by low-voltage-activated Caᵥ3-type calcium currents arising from the Caᵥ channels. Low-voltage-activated (LVA) calcium currents provide an important contribution to spike output patterns of neurons. Caᵥ3-typechannels are recognized as key determinants of LVA calcium-dependent responses, including low-threshold calcium spikes (LTS), unstable behavior, rebound depolarizations and augmentation of synaptic responses. Caᵥ3-typechannels are important to cell and circuit functions that range from sensory and pain transmission through thalamocortical sleep-wake cycles. The three isoforms of the Caᵥ3-type calcium channel, i.e., Caᵥ3.1, Caᵥ3.2, and Caᵥ3.3, can differ in their voltage-dependent and kinetic properties, demonstrating the potential to differentially affect spike output. In thalamus, differences in the distribution and kinetic properties of Caᵥ3-typecurrents have been shown to be capable of influencing the nature of oscillatory output of principal cells and inhibitory interneurons involved in the sleep-wake cycle, suggesting a selective distribution or modulation of Caᵥ3 channel isoforms over discrete regions of the cell axis.

There are many drugs currently available for treating neurological disorders. These drugs include, but are not limited to, anticonvulsants, antiepileptics, barbiturates, barbituric acid derivatives, anesthetic agents, tinnitus-treating agents, selective serotonin reuptake inhibitors, antidepressant agents, neuroleptic agents, antihypertensive agents, antipsychotic agents, calcium channel blockers, ACE inhibitors, and beta-blockers. However, many of such drugs are limited in their effectiveness and by their significant side effects. For example, some of these drugs are known to cause lightheadedness, depression insomnia, weakness, fatigue, and hallucinations, side-effects which severely limit their use with a human population. In particular, beta blockers, anticonvulsants, and benzodiazepines have been shown to be partially beneficial to some patients. However, beta blockers can cause changes in blood pressure and heart rate, and are contraindicated in patients with heart block, asthma, and congestive heart failure.

1-Octanol is one of the compounds which acts as an inhibitor of the low-threshold calcium channel (LTCC) [also called the transient (T-type) calcium channel or Caᵥ3.1 channel] and the subsequent TCD in vivo. 1-Octanol reversibly reduces the low threshold calcium currents in thalamic neurons in the brain and inferior olivary (10) neurons in the medulla oblongata. Octanol is known to reduce IO oscillations in vitro and to prevent harmaline-induced tremor thalamic level, and to block and reduce experimentally generated tinnitus, central pain and induced psychotic states generated by NMDA hypofunction.

Ethanol has similar effects on drug-induced tremor and is known to reduce essential familial tremor (EFT) in humans. Conversely, 1-octanol has been used and tested in a clinical trial to reduce tremor in patients with Essential Tremor.

U.S. Pat. No. 4,897,426 discloses a method for blocking calcium channels using C₂-C₁₀ alkyl alcohols.

US pre-grant patent application publication US2010/0292344 discloses methods and compositions for treating thalamocortical dysrhythmia using C₂-C₁₀ alkyl alcohols.

These C₂-C₁₀ alkyl alcohols or alkanols (Capryl Alcohols—CAs) have a strong pungent taste and warm-feeling imparting effect. Their pungent taste is not pure but contains some bitterness, burning and tingling sensation. These alkanols require a special care for handling because of their strong stimulation properties.

Octanol is a straight-chain fatty alcohol with 8 carbon atoms. Octanol has a sharp taste and smell. The compound occurs naturally in some plants and fruits, notably in citrus. It has been approved by the FDA and the Council of Europe, as a flavoring agent in food and cosmetics, with an accepted daily intake of 1 mg/kg.

In addition, octanol and other CAs have number of undesired properties. Mainly, CAs solubility in water is markedly low. CAs are liquid and volatile. CAs display “orange peel”-like notes. However, they have very pronounced detergent and/or painting-solvent smell making it undesirable to administer.

CAs have anesthetic and inhibiting properties for taste, smell and trigeminal senses after moderate exposure (numbness of the tongue and throat; inability to taste and smell fully foods and fragrances for several minutes).

Furthermore, octanol is associated with liver toxicity at higher dosages and the formulations described in the art deliver octanol at dosage close to toxic levels.

Thus, there is a need for new compositions or formulations of CAs which will markedly reduce the undesired properties including toxicity, impure taste and smell, and also will increase the water solubility and stability.

### SUMMARY OF THE INVENTION

In a first aspect, the present invention provides composition, pharmaceutical compositions, and pharmaceutical formulations thereof, having potency, specificity and selectivity in the prevention, prophylaxis, and treatment of neurological diseases, including for instance thalamocortical dysrhythmia, neurogenic pain, obsessive-compulsive disorder, depression, panic disorder, Parkinson's disease, schizophrenia, rigidity, dystonia, tinnitus, tremor, epilepsy, and other related conditions described herein.

Accordingly, the invention provides pharmaceutical compositions and formulations to treat a neurological disorder comprising capryl alcohol and cyclodextrin.

The invention further provides novel compositions and formulations comprising C₂-C₁₀ alkanol and cyclodextrin. Specifically, the invention provides novel compositions and formulations comprising octanol and cyclodextrin. More specifically, the invention provides novel compositions and formulations comprising 1-octanol and cyclodextrin. Furthermore, the invention provides novel compositions and formulations of caged or inclusion or clathrate compound comprising 1-octanol and cyclodextrin.

Accordingly, the invention provides pharmaceutical composition or formulation comprising:

a) cyclodextrin; and

b) a C₈ alcohol.

The invention further provides pharmaceutical compositions to treat neurological diseases or disorders comprising:

a) cyclodextrin; and

b) a C₈ alcohol.

The invention further provides pharmaceutical compositions or formulations comprising cyclodextrin and C₂-C₁₀ alkanol useful to treat neurological diseases or disorders.

In one particular embodiment, with respect to the pharmaceutical composition or formulation, the C₈ alcohol is 1-octanol.

In one particular embodiment, with respect to the pharmaceutical composition or formulation, the cyclodextrin is α-cyclodextrin, β-cyclodextrin, γ-cyclodextrin, branched cyclodextrin, glucosyl cyclodextrin, diglucosyl cyclodextrin, maltosyl cyclodextrin, dimaltosyl cyclodextrin, or hydroxypropyl cyclodextrin.

In another aspect, the invention provides low dosage formulations comprising 1-octanol. In one particular embodiment the amount of octanol administered is from 0.01 to 0.1 mg/kg. In another particular embodiment, the low dosage formulation is delivered using an upper body delivery method.

In another aspect, pharmaceutical compositions are provided comprising a compound of the invention, and a pharmaceutical carrier, excipient or diluent. The pharmaceutical composition can comprise one or more of the compounds described herein.

It will be understood that compounds of the present invention useful in the pharmaceutical compositions and treatment methods disclosed herein, can be pharmaceutically acceptable as prepared and used.

In a second aspect, the present invention provides methods for preventing, treating or ameliorating neurological disorders using the pharmaceutical compositions or formulation of the invention. In one particular embodiment, the disorder is thalamocortical dysrhythmia disorder.

In a further aspect, the present invention provides a use of pharmaceutical composition or formulations of the invention in oral mucosal delivery compositions.

In addition to the methods of treatment, the present invention extends to the use of any of the compounds or compositions described herein for the preparation of medicaments that may be administered for such treatments, as well as to such compounds for the treatments disclosed and specified.

Accordingly, it is a principal object of the invention to provide pharmaceutical compositions or formulations comprising 1-octanol and cyclodextrin effective to treat certain neurological disorders including, for instance, thalamocortical dysrhythmia disorder.

A further object of the invention is to provide formulations comprising a low dosage of 1-octanol.

A still further object of the invention is to provide a method for reducing symptoms associated with a neurological disorder such as thalamocortical dysrhythmia disorder.

Other objects and advantages will become apparent to those skilled in the art from a consideration of the ensuing detailed description which proceeds with reference to the following illustrative drawings.

### BRIEF DESCRIPTION OF THE FIGURES

FIG. 1: Effect of Octanol on Power Spectra. In most of the subjects there was an increase and broadening of the power spectra in the low alpha range. Top. Power spectra before and after octanol. Bottom. Same as in A at an expanded scale showing broadening of the alpha frequency range peak.

FIG. 2: Change in Mean Spectral Energy Difference in Mean Spectral Energy at five times after octanol for all subjects in the two frequency ranges showing the strongest change (grand mean, s.e.m). In spite of the variation there was a clear increase in the alpha range and decrease in delta range.

FIG. 3: Increase in Alpha Rhythm Power. The change in the power spectrum is shown here. The spectrum before octanol (black traces) and at 5 times after octanol (blue) are superimposed. The difference between these traces is shown in red. The difference in the mean spectral energy (MSE) before and after octanol between 8.5-10.4 Hz is plotted as a function of time after octanol showing the increased power (in dB). As in most subjects, there was no change at lower frequencies.

FIG. 4: Localization of Alpha Rhythm ICA was calculated to localize independent sources of brain activity before and after octanol. Most of the components were in the alpha frequency range, two are shown here before and 90 minutes after octanol. Localization of the alpha rhythms to the posterior part of the brain was not affected by octanol. Note the similarities between those before and after octanol administration.

FIG. 5: Decrease in Delta Rhythm Power Some subjects showed peaks at low frequencies as well as an alpha rhythm peak. In the example shown here there were two peaks in the delta frequency range (green line) and a small peak in the theta range (4-6 Hz). The delta range peaks were reduced after self-administration of octanol, while the alpha range peak increased (red traces). The MSE changes for 2.5-4.4 Hz (green lines and bars) and 8.5-10.4 Hz (purple lines and bars) are shown.

FIGS. 6A-6D: Localization of Delta & Alpha Rhythms after octanol administration. A&B. Low frequency sources. FIG. 6A. Component with sharp peak in the delta range and peak in theta range was localized to bilateral mesial orbital frontal cortex. FIG. 6B. Two hours after octanol there were no components with delta range peaks. The component shown does have theta range activity. This was chosen because its location was similar to that seen before octanol administration. However, the activity is not as strong as before octanol administration. FIG. 6C and FIG. 6D. The number of components in the alpha range increased after octanol administration, but their localization was very similar to that before octanol administration.

FIG. 7: No Change in Power. As shown in this example, low frequency peaks in some subjects were not affected by octanol. Quite similar sources in low frequencies (left) and with alpha peaks (right) were very similar as were their localizations. This is very different from those low frequency peaks that were reduced or eliminated by octanol indicating a different underlying generating mechanism.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties forth and defined below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

‘Compounds of the present invention’, and equivalent expressions, are meant to embrace the compounds as hereinbefore described, and in particular, compounds according to any of the formulae herein recited and/or described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Therapeutically effective amount’ means the amount of a compound that, when administered to a subject for treating a disease, is sufficient to effect such treatment for the disease. The ‘therapeutically effective amount’ can vary depending on the compound, the disease and its severity, and the age, weight, etc., of the subject to be treated.

‘Preventing’ or ‘prevention’ refers to a reduction in risk of acquiring or developing a disease or disorder (i.e., causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

‘Compounds of the present invention’, and equivalent expressions, are meant to embrace compounds of the Formula (e) as herein described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers, respectively). A chiral compound can exist as either individual enantiomer or as a mixture thereof. A mixture containing equal proportions of the enantiomers is called a ‘racemic mixture’.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure 5-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compositions, Formulations and Methods

In a first aspect, the present invention provides compositions, pharmaceutical compositions, and pharmaceutical formulations thereof, having potency, specificity and selectivity in the prevention, prophylaxis, and treatment of neurological diseases, including for instance thalamocortical dysrhythmia, neurogenic pain, obsessive-compulsive disorder, depression, panic disorder, Parkinson's disease, schizophrenia, rigidity, dystonia, tinnitus, tremor, epilepsy, and other related conditions described herein.

Accordingly, the invention provides pharmaceutical compositions and formulations to treat a neurological disorder comprising capryl alcohol and cyclodextrin.

The invention further provides novel compositions and formulations comprising C₂-C₁₀ alkanol and cyclodextrin. Specifically, the invention provides novel compositions and formulations comprising C₂-C₁₀ alkanol and cyclodextrin. More specifically, the invention provides novel compositions and formulations comprising C₂-C₁₀ alkanol and cyclodextrin. Furthermore, the invention provides novel compositions and formulations of caged or inclusion or clathrate compound comprising C₂-C₁₀ alkanol and cyclodextrin.

The invention further provides novel compositions and formulations comprising octanol and cyclodextrin. Specifically, the invention provides novel compositions and formulations comprising octanol and cyclodextrin. More specifically, the invention provides novel compositions and formulations comprising 1-octanol and cyclodextrin. Furthermore, the invention provides novel compositions and formulations of caged or inclusion or clathrate compound comprising octanol and cyclodextrin. Furthermore, the invention provides novel compositions and formulations of caged or inclusion or clathrate compound comprising 1-octanol and cyclodextrin.

Accordingly, the invention provides pharmaceutical composition or formulation comprising:

a) cyclodextrin; and

b) a C₈ alcohol.

The invention further provides pharmaceutical compositions or formulations to treat neurological diseases or disorders comprising:

a) cyclodextrin; and

b) a C₈ alcohol.

The invention further provides pharmaceutical compositions or formulations comprising cyclodextrin and C₂-C₁₀ alkanol useful to treat neurological diseases or disorders.

The invention further provides a pharmaceutical composition or formulation comprising an inclusion or caged compound comprising cyclodextrin and a C₂-C₁₀ alcohol.

In one embodiment, the inclusion compound is prepared by mixing cyclodextrin and a C₂-C₁₀ alcohol.

In one embodiment, the inclusion compound is prepared by mixing cyclodextrin and a C₂-C₁₀ alcohol and is in powdered form.

In one embodiment, the inclusion compound is cyclodextrin encapsulated C₂-C₁₀ alcohol.

The invention further provides a pharmaceutical composition or formulation comprising an inclusion or caged compound comprising cyclodextrin and a C₈ alcohol.

In one embodiment, with respect to the pharmaceutical composition or formulation comprising an inclusion or caged compound, the inclusion compound is prepared by mixing cyclodextrin and a C₈ alcohol. In a particular embodiment, the C₈ alcohol is 1-octanol.

In one embodiment, with respect to the pharmaceutical composition or formulation comprising an inclusion or caged compound, the inclusion compound is prepared by mixing cyclodextrin and a C₈ alcohol and is in a powdered form. In a particular embodiment, the C₈ alcohol is 1-octanol.

In one embodiment, with respect to the pharmaceutical composition or formulation comprising an inclusion or caged compound, the inclusion compound is cyclodextrin encapsulated C₈ alcohol. In a particular embodiment, the C₈ alcohol is 1-octanol.

In one embodiment, with respect to the pharmaceutical composition or formulation of the invention, the cyclodextrin is α-cyclodextrin, β-cyclodextrin, γ-cyclodextrin, branched cyclodextrin, glucosyl cyclodextrin, diglucosyl cyclodextrin, maltosyl cyclodextrin, dimaltosyl cyclodextrin, or hydroxypropyl cyclodextrin. In another embodiment, the cyclodextrin is α-cyclodextrin, β-cyclodextrin, or γ-cyclodextrin. In yet another embodiment, the cyclodextrin is branched cyclodextrin, glucosyl cyclodextrin, diglucosyl cyclodextrin, maltosyl cyclodextrin, dimaltosyl cyclodextrin. In a further embodiment, the cyclodextrin is hydroxypropyl cyclodextrin. In a particular embodiment, the cyclodextrin is β-cyclodextrin, or γ-cyclodextrin or hydroxypropyl cyclodextrin.

In one embodiment, with respect to the pharmaceutical composition or formulation of the invention, the C₈ alcohol is 1-octanol, 2-octanol or 3-octanol. In one particular embodiment the C₈ alcohol is 1-octanol.

In one embodiment, with respect to the pharmaceutical composition or formulation of the invention, the pharmaceutical composition or formulation further comprises natural vanilloid extracts.

In one embodiment, with respect to the pharmaceutical composition or formulation of the invention, the pharmaceutical composition or formulation further comprises one or more oil based compounds. In one embodiment, the oil based compound is castor oil. In a further embodiment, it is cremophor.

In one embodiment, with respect to the pharmaceutical composition or formulation of the invention, the pharmaceutical composition or formulation further comprises one or more oil based compounds selected from castor oil and hydrogenated castor oil.

In one embodiment, with respect to the pharmaceutical composition or formulation of the invention, the pharmaceutical composition or formulation further comprises a carrier or adjuvant.

In one embodiment, with respect to the pharmaceutical composition or formulation of the invention, the pharmaceutical composition or formulation comprises a carrier and the carrier is a parenteral carrier.

In one embodiment, with respect to the pharmaceutical composition or formulation of the invention, the pharmaceutical composition or formulation comprises a carrier and the carrier is an oral carrier.

In one embodiment, with respect to the pharmaceutical composition or formulation of the invention, the pharmaceutical composition or formulation comprises a carrier and the carrier is a topical carrier.

In one embodiment, with respect to the pharmaceutical composition or formulation of the invention, the pharmaceutical composition or formulation comprises a carrier and the carrier is a transmucosal carrier.

In one embodiment, with respect to the pharmaceutical composition or formulation of the invention, the pharmaceutical composition or formulation comprises a carrier and the carrier is a transmucosal carrier selected from nasal, buccal, sublingual, vaginal, ocular, and rectal.

In one embodiment, with respect to the pharmaceutical composition or formulation of the invention, the pharmaceutical composition or formulation comprises an inclusion or caged compound of the invention and vanilla-bean essential oil. In another embodiment, the formulation further comprises ethanol. In another embodiment, the formulation further comprises castor oil.

In one embodiment, with respect to the pharmaceutical composition or formulation of the invention, the pharmaceutical composition or formulation is in the form of a chewing gum. In one embodiment, the chewing gum comprises an inclusion or caged compound of the invention. In another embodiment, the chewing gum further comprises a flavoring agent.

In one embodiment, the flavoring agent is vanilla flavor agent. In another embodiment, the chewing gum further comprises thick malt syrup. In another embodiment, the chewing gum further comprises gum base resin. In another embodiment, the chewing gum further comprises sugar-sucrose mix. In one particular embodiment, the mix is in a 1:1 ratio.

In one embodiment, with respect to the pharmaceutical composition or formulation of the invention, the pharmaceutical composition or formulation is in the form of mouth-meltable pills, or homeopathic pills.

In one embodiment, with respect to the pharmaceutical composition or formulation of the invention, the pharmaceutical composition or formulation is in the form of a mouthwash. In one embodiment, the mouthwash comprises the caged or inclusion compound of the invention and ethanol. In one embodiment, the mouthwash comprises sodium lauryl sulfate, glycerine, vanilla-bean essential oil extract, coloring matter and/or flavoring.

In one embodiment, with respect to the pharmaceutical composition or formulation of the invention, the pharmaceutical composition or formulation is in the form of transmucosal formulation. In one embodiment, the formulation is in the form of lingual spray, sublingual pills, or mouth dissolving pills.

In one embodiment, with respect to the pharmaceutical composition or formulation of the invention, the pharmaceutical composition or formulation is prepared as a slow release formulation.

In one embodiment, with respect to the pharmaceutical composition or formulation of the invention, the pharmaceutical composition or formulation is in the form of a liquid spray.

In one embodiment, the liquid spray comprises the caged or inclusion compound of the invention. In another embodiment, the liquid spray further comprises castor oil. In another embodiment, the formulation further comprises coffee syrup, sucrose, sucralose, vanilla, vanilla extract, ethanol and octanol.

In one embodiment, with respect to the pharmaceutical composition or formulation of the invention, the pharmaceutical composition or formulation is delivered using methods selected from peroral, topical, and transmucosal.

In one embodiment, with respect to the pharmaceutical composition or formulation of the invention, the pharmaceutical composition or formulation is delivered by an inhalation route.

In one embodiment, with respect to the pharmaceutical composition or formulation of the invention, the pharmaceutical composition or formulation is delivered by an upper body delivery method or system. In one embodiment, the upper body delivery method or system is nasal or lingual spray.

In one embodiment, with respect to the pharmaceutical composition or formulation of the invention, the pharmaceutical composition or formulation is incorporated in mouth meltable pills, mouth meltable strips, sublingual micro pills, gum, candy, ointment, gel, systemic lotion, systemic gel, massage cream, massage gel, mouth meltable pills, capsules, homeopathic pills, hair lotion, or mouth wash.

In one embodiment, with respect to the pharmaceutical composition or formulation of the invention, the pharmaceutical composition or formulation is delivered using a lingual spray, sublingual pill, mouth dissolving strip or chewing gum.

In one embodiment, with respect to the pharmaceutical composition or formulation of the invention, the pharmaceutical composition or formulation is delivered using a slow sustained release method.

In one embodiment, with respect to the pharmaceutical composition or formulation of the invention, the pharmaceutical composition or formulation is to treat neurological disorder; and the neurological disorder is any neurological disorder described herein. In one embodiment the neurological disorder is thalamocortical dysrhythmia disorder.

In one embodiment, with respect to the pharmaceutical composition or formulation of the invention, the pharmaceutical composition or formulation is a low dosage formulation. In one embodiment the amount of 1-octanol administered is in the range of 0.001 to 1 mg/kg. In one particular embodiment, the amount of 1-octanol administered is in the range of 0.01 to 0.1 mg/kg.

In one embodiment, with respect to the pharmaceutical composition or formulation of the invention, the pharmaceutical composition or formulation is used as a nootropic agent.

In a second aspect, the present invention provides a method of treating a neurological disorder in a mammal in need thereof, the method comprising administering to the mammal a therapeutically effective amount of the pharmaceutical composition or formulation of the invention. In one embodiment the neurological disorder is tremor or Parkinson's tremor. In one embodiment the neurological disorder is other than tremor or Parkinson's tremor.

In another aspect, the present invention provides a method of treating a thalamocortical dysrhythmia disorder that is not tremor or Parkinson's tremor in a mammal in need thereof, the method comprising administering to the mammal a therapeutically effective amount of the pharmaceutical composition or formulation of the invention.

In one embodiment, with respect to the method of the invention, the thalamocortical dysrhythmia disorder is petit mal epilepsy.

In another embodiment, with respect to the method of the invention, the thalamocortical dysrhythmia disorder is tinnitus.

In another embodiment, with respect to the method of the invention, the thalamocortical dysrhythmia disorder is neurogenic pain.

In another embodiment, with respect to the method of the invention, the thalamocortical dysrhythmia disorder is obsessive-compulsive disorder.

In another embodiment, with respect to the method of the invention, the thalamocortical dysrhythmia disorder is Tourettes disease.

In another embodiment, with respect to the method, the thalamocortical dysrhythmia disorder is chronic depression.

In another embodiment, with respect to the method of the invention, the thalamocortical dysrhythmia disorder is autism or Asperger's syndrome.

In another embodiment, with respect to the method of the invention, the thalamocortical dysrhythmia disorder is schizoaffective psychosis.

In another embodiment, with respect to the method of the invention, the thalamocortical dysrhythmia disorder is Parkinson's disorder.

In another embodiment, with respect to the method of the invention, the thalamocortical dysrhythmia disorder is Migraine.

In another embodiment, with respect to the method of the invention, the thalamocortical dysrhythmia disorder is Absence Epilepsy.

In another embodiment, with respect to the method of the invention, the thalamocortical dysrhythmia disorder is restless leg syndrome.

In a second aspect, the present invention provides a method of treating neurogenic pain, obsessive-compulsive disorder, depression, panic disorder, Parkinson's disease, schizophrenia, rigidity, dystonia, tinnitus, tremor, and epilepsy in a mammal in need thereof, the method comprising administering to the mammal a therapeutically effective amount of the pharmaceutical composition or formulation of the invention.

In one embodiment, the tremor is essential tremor. In another embodiment, the tremor is essential familial tremor (EFT).

In another aspect, the present invention provides a method of improving alertness and attentiveness in the healthy humans, the method comprising administering to the mammal a therapeutically effective amount of pharmaceutical composition or formulation of the invention.

In another aspect, the present invention provides a method of improving focal concentration capability in the healthy humans, the method comprising administering to the mammal a therapeutically effective amount of pharmaceutical composition or formulation of the invention.

In another aspect, the present invention provides a method of enhancing cognition and concentration in healthy adults, the method comprising administering to the mammal a therapeutically effective amount of pharmaceutical composition or formulation of the invention.

In another aspect, the present invention provides a method to aid meditative states in the healthy adults, the method comprising administering to the mammal a therapeutically effective amount of pharmaceutical composition or formulation of the invention.

In another aspect, the present invention provides a method of promoting nootropic activity in a patient comprising administering to said patient a therapeutically effective amount of pharmaceutical composition or formulation of the invention.

In one embodiment, with respect to the method, the therapeutically effective amount administered is from about 0.001 mg/kg body weight to about 100 mg/kg body weight of the mammal.

In another embodiment, with respect to the method, the therapeutically effective amount administered is from about 0.1 mg/kg body weight to about 10 mg/kg body weight of the mammal.

In another embodiment, with respect to the method, the therapeutically effective amount administered is from about 0.3 mg/kg body weight to about 3.0 mg/kg body weight of the mammal.

In another embodiment, with respect to the method, the therapeutically effective amount administered is about 1.0 mg/kg body weight of the mammal.

The invention further provides a method of improving the taste of a formulation containing C₈ alcohol, wherein the method comprises encapsulating the C₈ alcohol in cyclodextrin. In one particular embodiment, the C₈ alcohol is 1-octanol. In another embodiment, the cyclodextrin is α-cyclodextrin, β-cyclodextrin, γ-cyclodextrin, branched cyclodextrin, glucosyl cyclodextrin, diglucosyl cyclodextrin, maltosyl cyclodextrin, dimaltosyl cyclodextrin, or hydroxypropyl cyclodextrin. In a particular embodiment, the cyclodextrin is β-cyclodextrin.

In another embodiment, with respect to the method of the invention, the method further comprises addition of vanilloid extracts to the formulation.

The invention further provides a use of a pharmaceutical composition or formulation of the invention, in oral mucosal delivery compositions, skin preparations for external use, bathing compositions, in foods, or in beverages.

The invention further provides a use of a pharmaceutical composition or formulation of the invention, in skin patches, gels, or balms.

The invention further provides a composition comprising an inclusion or caged compound comprising cyclodextrin and a C₈ alcohol.

The invention further provides a composition comprising an inclusion or caged compound comprising cyclodextrin and 1-octanol.

The invention further provides a low dosage formulation comprising 1-octanol.

In one embodiment, with respect to the low dosage formulation of the invention, the effective amount of 1-octanol administered is from 0.01 to 1 mg/kg. In another embodiment the dose is <0.1 mg/kg. In one particular embodiment, the amount is 0.01 to 0.1 mg/kg.

In one embodiment, with respect to the composition or formulation of the invention, the composition or formulation is incorporated in mouth meltable pills, mouth meltable strips, sublingual micro pills, gum, candy, ointment, gel, systemic lotion, systemic gel, massage cream, massage gel, mouth meltable pills, capsules, homeopathic pills, hair lotion, or mouth wash.

In one embodiment, with respect to the composition or formulation of the invention, the composition or formulation is delivered using a lingual spray, sublingual pill, mouth dissolving strip or chewing gum.

In one embodiment, with respect to the composition or formulation of the invention, the composition or formulation is delivered using a slow sustained release method.

In one embodiment, with respect to the composition or formulation of the invention, the pharmaceutical composition or formulation is delivered by an inhalation route or an upper body delivery method.

In one embodiment, with respect to the composition or formulation of the invention, the pharmaceutical composition or formulation is delivered using a spray.

The invention further provides a method of preparation of an inclusion compound of a C₈ alcohol and cyclodextrin.

In one embodiment, with respect to the method of preparation, the method comprises adding the C₈ alcohol to a solution of cyclodextrin in water.

In another embodiment, with respect to the method of preparation, the method comprises dissolving cyclodextrin in water and adding the C₈ alcohol to the resulting solution.

In another embodiment, with respect to the method of preparation, the method comprises the steps of
i) dissolving cyclodextrin in water
ii) adding C₈ alcohol
iii) stirring for 0.1-24 h and
iv) collecting the precipitated inclusion compound by filtration.

In a more particular aspect, the present invention provides a low dose formulation comprising cyclodextrin and 1-octanol, wherein, when administered, the effective dose of 1-octanol is less than 0.1 mg/kg. In one embodiment the low dose formulation comprises a caged or inclusive compound of 1-octanol and cyclodextrin. In one embodiment, the low dose formulation is delivered using an upper body delivery method, for instance, a lingual or nose spray.

In a further particular aspect, the present invention provides a method of using 1-octanol as a nootropic drug. In one embodiment, the method comprises administering 1-octanol in a low dosage formulation.

In one embodiment, with respect to the pharmaceutical composition of the invention, the carrier is a parenteral carrier. In one embodiment, with respect to the pharmaceutical composition of the invention, the carrier is an oral carrier. In one embodiment, with respect to the pharmaceutical composition of the invention, the carrier is a topical carrier.

In a particular aspect, the present invention provides a novel form of C₂-C₁₀ alkanol. In one embodiment, with respect to the novel form of C₂-C₁₀ alkanol, the novel form is an encapsulated C₂-C₁₀ alkanol in cyclodextrin. In one particular embodiment, the novel form is a powdered or solid form.

In a particular aspect, the present invention provides a novel form of octanol. In one embodiment, with respect to the novel form of octanol, the novel form is an encapsulated octanol in cyclodextrin. In one particular embodiment, the novel form is a powdered or solid form.

In a particular aspect, the present invention provides a novel form of 1-octanol. In one embodiment, with respect to the novel form of 1-octanol, the novel form is an encapsulated-1-octanol in cyclodextrin. In one particular embodiment, the novel form is a powdered or solid form.

In a particular aspect, the present invention provides a novel concept of masking the undesired properties of capryl alcohols. In one particular embodiment, the present invention provides a novel concept of masking the undesired properties of 1-octanol.

In one aspect, the invention provides pharmaceutical formulation of the inclusion compound of octanol in cyclodextrin. In one embodiment, the cyclodextrin is β-CD, G1-β-CD, or γ-CD. In one embodiment, octanol is 1-octanol. In one embodiment, the formulation further comprises vanilla-bean essential oil. In one embodiment, the formulation is in chewing gum, mouth-meltable pills, homeopathic pills, hair lotions, mouth wash, lingual spray, sublingual pills, mouth dissolving strips or liquid spray form.

In one aspect, the invention provides preparation of chewing gum, mouth-meltable pills, homeopathic pills, hair lotions, mouth wash, lingual spray, sublingual pills, mouth dissolving strips and liquid spray comprising the formulation of the inclusion compound of octanol in cyclodextrin. In one embodiment, the cyclodextrin is β-CD, G1-β-CD, or γ-CD. In one embodiment, octanol is 1-octanol.

Additional embodiments within the scope of the present invention are set forth in non-limiting fashion elsewhere herein and in the examples. It should be understood that these examples are for illustrative purposes only and are not to be construed as limiting this invention in any manner.

Certain compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ or C₁-C₆alkyl, C₂-C₈ alkenyl, aryl, substituted aryl, and arylalkyl esters of the compounds of the invention.

### Pharmaceutical Compositions

When employed as pharmaceuticals, the compounds of the invention are typically administered in the form of a pharmaceutical composition. Such compositions can be prepared in a manner well known in the pharmaceutical art and comprise at least one active compound. In certain embodiments, the pharmaceutical composition may comprise a compound of the invention in combination with one or more compounds or compositions of like therapeutic utility and effect.

Generally, the compounds of the invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the invention can be administered by a variety of routes including oral, rectal, transdermal, subcutaneous, intravenous, intramuscular, and intranasal. Depending on the intended route of delivery, the compounds of this invention are preferably formulated as injectable, oral or intranasal compositions or as salves, as lotions or as patches, all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Intranasal compositions are typically mucoadhesive temperature-mediated in situ gel formulations that may use chitosan and hydroxylpropyl methylcellulose which enhance intranasal fixation and absorption producing transport into the central nervous system. See, for example, Khan, S., Patil, K., Bobade, N., Yeole, P., Gaikwad, R. 2010. Formulation of intranasal mucoadhesive temperature-mediated in situ gel containing ropinirole and evaluation of brain targeting efficiency in rats. J Drug Target 18, 223-234.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream, for example, with an oil-in-water cream base. Such transdermal formulations are well-known in the art and generally include additional ingredients to enhance the dermal penetration and stability of the active ingredients or the formulation. All such known transdermal formulations and ingredients are included within the scope of this invention.

The compositions of this invention can also be administered by a transdermal device. Accordingly, transdermal administration can be accomplished using a patch of either the reservoir or porous membrane type, or of a solid matrix variety.

The above-described components for orally administrable, injectable or topically administrable compositions are merely representative. Other materials as well as processing techniques and the like are set forth in Part 8 of Remington's The Science and Practice of Pharmacy, 21^{st} edition, 2005, Publisher: Lippincott Williams & Wilkins, which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

The particular pungency masker oil (castor oil, cremophor) and other taste and trigeminal sensation-improving/masking ingredients (vanilla oil, toffee-nut flavors) employed will vary depending upon the particular taste and feel desired. Those skilled in the art can select and customize these types of ingredients to provide the desired results.

Additional conventional components may be added as in mouthwashes of the prior art. Whereas some ethanol containing mixtures have a pH of about 7.0, reduction of the ethanol level may requires the addition of acidic preservatives, such as sorbic or benzoic acid, which reduce pH levels. Buffer systems are then necessary to control the pH of the composition at optimal levels. This is generally accomplished through the addition of a weak acid and its salt or a weak base and its salt. Useful systems have been found to be and to include, sodium benzoate and benzoic acid in amounts of from about 0.01% to about 4.0% w/v, and sodium citrate and citric acid in amounts of from about 0.001% to about 0.2% w/v. Preferably the buffers are incorporated in amounts that maintain the pH at levels of from about 3.5 to about 9, and more preferably from about 4.0 to 7.0.

Other conventional ingredients may be used in the ethanol-based octanol compositions of this invention, including those known and used in the art. For example, humectants such as polyethylene glycol may be added as an additional solubilizer for the flavor/active-natural compound oils (vanilla oil) and to also provide texture to the composition. These are incorporated in amounts of from about 0.3% w/v to about 5.0% w/v, and enhance the lubricous mouthfeel of the composition as it is used and to provide a refreshing, moist, organoleptic feeling thereafter. Glycerin may be incorporated in amounts of from about 1.0% w/v to about 16.0% w/v, and preferably in an amount of about 7.5% w/v. Sweeteners such as sucralose and the like may be added for better taste and bitter-taste-masking, in amounts of from about 0.005% w/v to about 1.0% w/v.

The skilled person in the food and beverage industry knows of the critical role flavor plays in the appreciation of foods and beverages. Many food products, such as cocoa, chocolate, coffee, caramel, nuts, malt and the like have flavor quality that is referred to in the language of this art as “roasted brown”. In the following, the term “roasted brown” will be used to describe flavor properties of the mentioned food products as well as of food products having similar flavor characteristics. The term flavor comprises in the following flavor, aroma and taste, with marked “taste-masking” properties.

Mixtures of such flavors with the present composition or formulation, impart the pungent-like solvent-like taste and smell of octanol to chocolate, cocoa and other products with roasted brown flavor. These flavors are also useful for enhancing or modifying the flavor profile to other masking deep flavors such as roasted brown flavors, like cocoa, chocolate, coffee, caramel, toffee, butterscotch, roasted and regular nuts, like hazelnuts, almonds, walnuts, chestnuts, macadamia, coconut, roasted butter, condensed milk, malt and the like.

### Formulation 1

### Tablets

The components of the pharmaceutical composition of the invention may be admixed as a dry powder with a dry gelatin binder in an approximate 1:2 weight ratio. A minor amount of magnesium stearate is added as a lubricant. The mixture may be formed into 240-270 mg tablets (80-90 mg of active compound per tablet) in a tablet press.

### Formulation 2

### Capsules

The components of the pharmaceutical composition of the invention may be admixed as a dry powder with a starch diluent in an approximate 1:1 weight ratio. The mixture may be filled into 250 mg capsules (125 mg of active compound per capsule).

### Formulation 3

### Liquid

The components of the pharmaceutical composition of the invention (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then be added to produce a total volume of 5 mL.

### Formulation 4

### Tablets

The components of the pharmaceutical composition of the invention may be admixed as a dry powder with a dry gelatin binder in an approximate 1:2 weight ratio. A minor amount of magnesium stearate is added as a lubricant. The mixture may be formed into 450-900 mg tablets (150-300 mg of active compound) in a tablet press.

### Formulation 5

### Injection

The components of the pharmaceutical composition of the invention may be dissolved or suspended in a buffered sterile saline injectable aqueous medium to a concentration of approximately 5 mg/mL.

### Formulation 6

### Topical

Stearyl alcohol (250 g) and a white petrolatum (250 g) may be melted at about 75° C. and then a mixture of a the components of the pharmaceutical composition of the invention (50 g, in proper ratio) methylparaben (0.25 g), propylparaben (0.15 g), sodium lauryl sulfate (10 g), and propylene glycol (120 g) dissolved in water (about 370 g) is added and the resulting mixture is stirred until it congeals.

### Formulation 7

### Intranasal

Temperature-mediated in situ gelling systems may be prepared by dissolving the components of the pharmaceutical composition of the invention, chitosan HCl (1% w/v) and HPMC (varying grades and concentrations) in 0.5% sodium chloride maintained at temperature ˜4° C. To the resulting solution 1 mL 0.282 M sodium β-glycerophosphate solution (ultimate concentration 8.8 wt %) may be added drop by drop with continuous stirring while maintaining the temperature below 10° C. using an ice bath. Benzalkonium chloride (0.05% w/v) is added and pH adjusted to 7.0 using 1 M NaOH. A final volume of 10 mL is achieved with 0.5% sodium chloride. The formulations are stored below 10° C.

### Methods of Treatment

The pharmaceutical compositions of the present invention may be used as therapeutic agents for the treatment of conditions in mammals. Accordingly, the compounds and pharmaceutical compositions of this invention find use as therapeutics for preventing and/or treating illnesses, diseases, ailments, etc. such as, but not limited to, neurological disorders, such as, for instance thalamocortical dysrhythmia.

In a method of treatment aspect, the invention provides a method of treating a mammal susceptible to or afflicted with a condition as recited above, which method comprises administering an effective amount of one or more of the pharmaceutical compositions described herein. In additional method of treatment aspects, the invention provides methods of treating a mammal susceptible to or afflicted with a neurological disorder, such as, for instance, thalamocortical dysrhythmia, the method comprises administering an effective condition-treating or condition-preventing amount of one or more of the pharmaceutical compositions just described.

As a further aspect of the invention there is provided the present compounds for use as a pharmaceutical especially in the treatment or prevention of the aforementioned conditions and diseases. The invention extends to the use of the present compounds in the manufacture of a medicament for the treatment or prevention of one of the aforementioned conditions and diseases.

Injection dose levels range from about 0.001 mg/kg/hour to at least 1 mg/kg/hour, all for from about 1 to about 120 hours and especially 24 to 96 hours. A preloading bolus of from about 0.01 mg/kg to about 1 mg/kg or more may also be administered to achieve adequate steady state levels.

For the prevention and/or treatment of long-term conditions, such as CNS conditions, the regimen for treatment is usually extended over many months or years, and consequently, oral dosing is preferred for patient convenience and tolerance. With oral dosing, one to five, particularly two to four, and typically three oral doses per day are representative regimens. Using these dosing patterns, each dose provides from about 0.001 to about 2 mg/kg of the compound of the invention, with preferred doses each providing from about 0.01 to about 1 mg/kg and especially about 0.01 to about 0.5 mg/kg. Transdermal doses are generally selected to provide similar or lower blood levels than are achieved using injection doses.

The pharmaceutical compositions of this invention can be administered as the sole active composition or they can be administered in combination with other agents, including other active ingredients and derivatives.

### General Synthetic Procedures

The components of the pharmaceutical compostions or formulations of the invention can be obtained from commercial sources or can be prepared from readily available starting materials using the following general methods and procedures. See, e.g., Synthetic Scheme, below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection, are all well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

For example, the compositions or formulations may be prepared by following methods known to one skilled in the art, and the product thereafter isolated and purified by known standard procedures. Such procedures include (but are not limited to) recrystallization, column chromatography or HPLC.

### Therapeutic Effects

Octanol has a complex pharmacological profile; and is active with a plethora of channels and receptors including Caᵥ, at different doses, as referenced in the literature.

For alcohol-sensitive GABA types, for example:

GABA receptors are implicated in tremors. Some benzodiazepines are efficient. Interestingly, octanol is a slight allosteric potentiator of GABA at low doses (1-10 μm), and becomes an allosteric inhibitor of GABA at a higher dose. Furthermore, octanol has the capability to be a direct agonist in a certain range of concentration and then auto-inhibitor by itself at a higher dosage (bell-curve) [Agonist and Potentiation Actions of n-Octanol on GABA_{A} Receptors Yasutaka Kurata, 1 William Marszalec, Jay Z. Yeh, and Toshio Narahashi, Mol Pharmacol. 1999 June; 55(6):1011-9].

Therefore, one can expect a synergistic slight ‘benzodiazepine-like’ effect of octanol at low dosage. One can also predict a ‘depressant effect’ on GABA pathways at higher concentrations of octanol.

### The effect of 1-Octanol on Spontaneous Brain Activity in Healthy Adults

As described above, 1-Octanol has ability to block T-type (Caᵥ3.1) calcium channels. These channels are a key element in the generation of low frequency oscillations (delta and theta ranges) such as those seen in thalamocortical dysrhythmia (TCD). Octanol reversibly reduces the low threshold calcium currents in thalamic [1] and inferior olivary (10) neurons [2], and reduces 10 oscillations [3] in vitro and prevents harmaline-induced tremor [4]. 1-Octanol has also been used to reduce tremor in patients with essential tremor [5,6]. Beyond essential tremor the effect of octanol in thalamocortical dysrhythmia (TCD) [7] is being studied as it has been shown to be effective in TCD in an animal model [8]. The contents of the cited references are hereby incorporated by reference in their entireties.

In addition, the changes in brain spectral activity following the absorption of the drug in healthy subjects suggest potential beneficial and/or appreciable effects of the drug on the healthy brain function. Thus, 1-octanol may be useful as a nootropic agent. Nootropics can be divided into several subsets depending on how they “positively” act on the mind and cognition. The word ‘cognition’ itself covers a wide range of facets of brain function, including learning, memory, attention and motivation. While many well-known nootropic drugs can be effective in increasing alertness/awareness (e.g. the family of stimulants/excitants: caffeine, amphetamines, modafinil), 1-octanol would not be expected to formally fall within that class of drugs as 1-octanol did not promote a significant increase of beta range activity as could be expected for increased alert/anxious states. 1-octanol significantly increased lower alpha band power, promoting of broadening and slight peak shift of the whole alpha-band spectrum, with a general increase in the normal alpha range activity. 1-octanol also had a tendency for potentiating higher theta range activity in some subjects; higher theta range activity has been associated with reports of meditative and creative states while theta range itself has also been reported abnormally elevated in some TCD. Finally, in some subjects an increase in gamma range activity was also noted; which can reflect certain cognitive function.

### Example 1

### Participants

The database comprises 29 healthy adults from 25-52 years of age (12 women and 17 men).

### MEG Recordings of Spontaneous Activity

Spontaneous activity was recorded using a 275 channel (CTF) MEG. Six 7-min recordings were made with the eyes closed. One recording was made before self-administration of 1-Octanol. The others were made at 15, 30, 60, 90 and 120 min after.

### Spectral Analysis

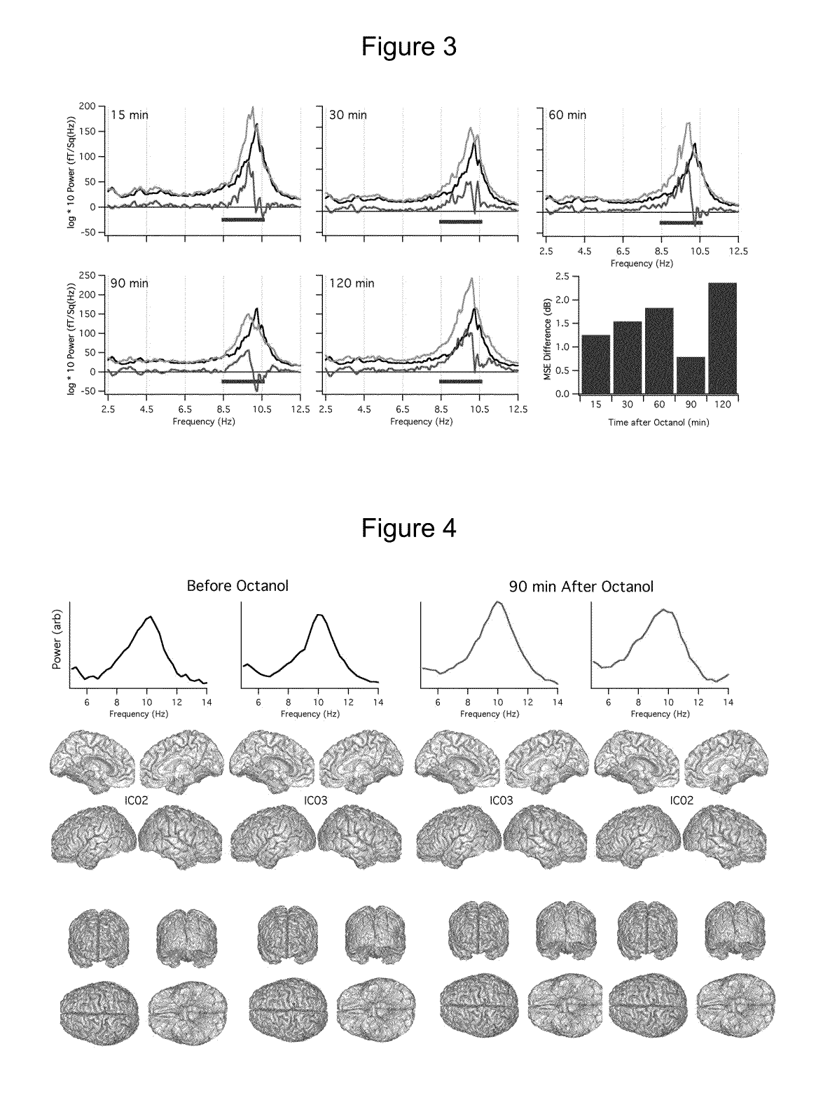

A multi-taper approach, which provides reduced-variance calculations of frequency, was used to generate frequency spectra from each set of recordings. Changes in the power spectrum after octanol was determined by first calculating the mean spectra energy (MSE) in 2 Hz frequency bands starting at 2.5 Hz. Logarithmic transformation was applied to the power data and then the mean spectral energy (MSE) was calculated for each band before and after octanol administration. The difference in MSE before and after octanol was then calculated for each participant and the grand mean calculated (FIG. 3).

### Octanol Preparation

Octanol is a straight chain fatty alcohol with 8 carbon atoms CH₃(CH₂)₇OH, and was formulated in an oral mucosal delivery format with appropriate organoleptic characteristics. It was self-administered at 0.1 mg/kg. Participants were instructed to use the spray as they would a breathe spray.

### ICA & Localization

Independent component analysis (ICA) was carried out on the sensor data using the Infomax EEGLab algorithm. The magnetic field described by the ICs was localized onto a volumetric source space constructed from a population-averaged MRI using a recursive weighted minimum norm algorithm.

### Questionnaire

A seven-scale questionnaire was used before and at 5-10 minutes and 120 minutes, respectively, after octanol administration to examine the level of relaxation, concentration, worry, sleepiness, stress, pain and the octanol taste.

### Results

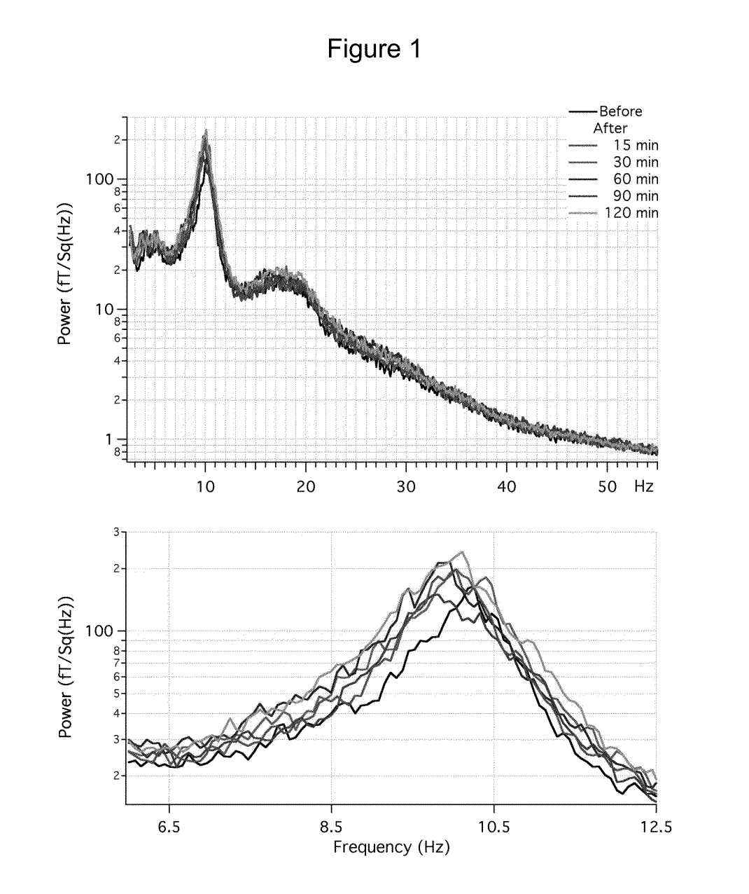

FIG. 1: Effect of Octanol on Power Spectra. In most of the subjects there was an increase and broadening of the power spectra in the low alpha range. Top. Power spectra before and after octanol administration. Bottom. Same as in A, at an expanded scale showing broadening of the alpha frequency range peak.

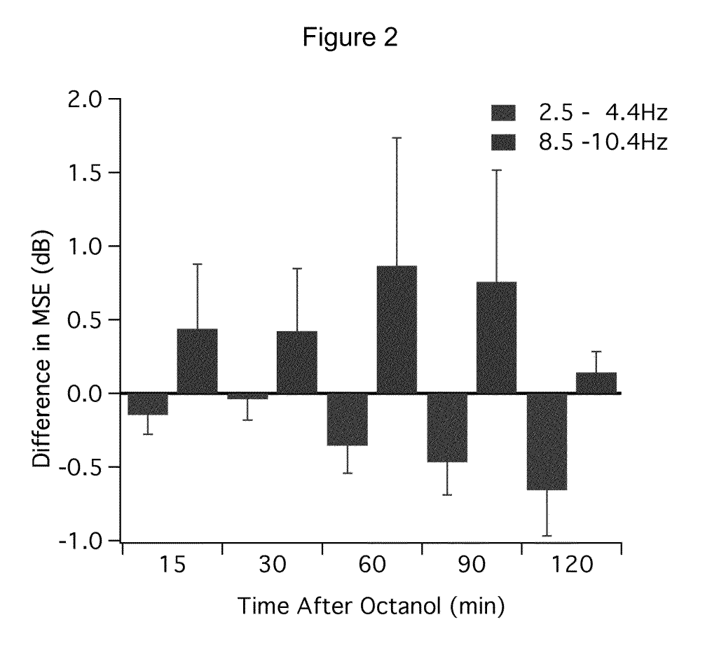

FIG. 2: Change in Mean Spectral Energy. Difference in Mean Spectral Energy at five times after octanol administration for all subjects in the two frequency ranges showing the strongest change (grand mean, s.e.m). In spite of the variation there was a clear increase in the alpha range and decrease at delta.

FIG. 3: Increase in Alpha Rhythm Power. The change in the power spectrum is shown here. The spectrum before octanol administration (black traces) and at 5 times after octanol administration (blue) are superimposed. The difference between these traces is shown in red. The difference in the mean spectral energy (MSE) before and after octanol administration between 8.5-10.4 Hz is plotted as a function of time after octanol administration showing the increased power (in dB). As in most subjects, there was no change at lower frequencies

FIG. 4: Localization of Alpha Rhythm. ICA was calculated to localize independent sources of brain activity before and after octanol administration. Most of the components were in the alpha frequency range. Two are shown here before and 90 minutes after octanol administration. Localization of the alpha rhythms to the posterior part of the brain was not affected by octanol. Note the similarities between those before and after octanol administration.

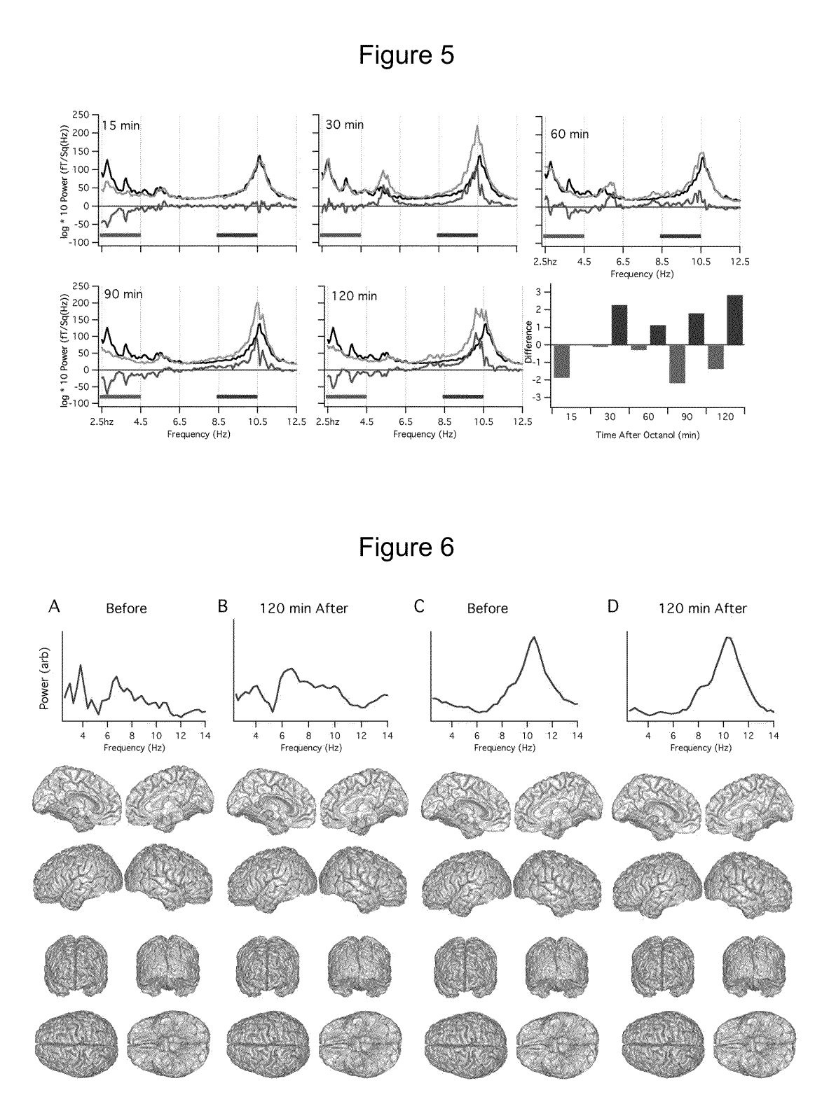

FIG. 5: Decrease in Delta Rhythm Power. Some subjects showed peaks at low frequencies as well as an alpha rhythm peak. In the example shown here there were two peaks in the delta frequency range (green line) and a small peak in the theta range (4-6 Hz). The delta range peaks were reduced after self-administration of octanol, while the alpha range peak increased (red traces). The MSE changes for 2.5-4.4 Hz (green lines and bars) and 8.5-10.4 Hz (purple lines and bars are shown

FIG. 6: Localization of Delta & Alpha Rhythms after octanol administration. A&B. Low frequency sources. A. Component with sharp peak in the delta range and peak in theta range was localized to bilateral mesial orbital frontal cortex. B. Two hours after octanol administration there were no components with delta range peaks. The component shown does have theta range activity. This was chosen because its location was similar to that seen before octanol administration. However, the activity is not as strong as before octanol administration. C&D The number of components in the alpha range increased after octanol administration, but their localization was very similar to that before octanol administration.

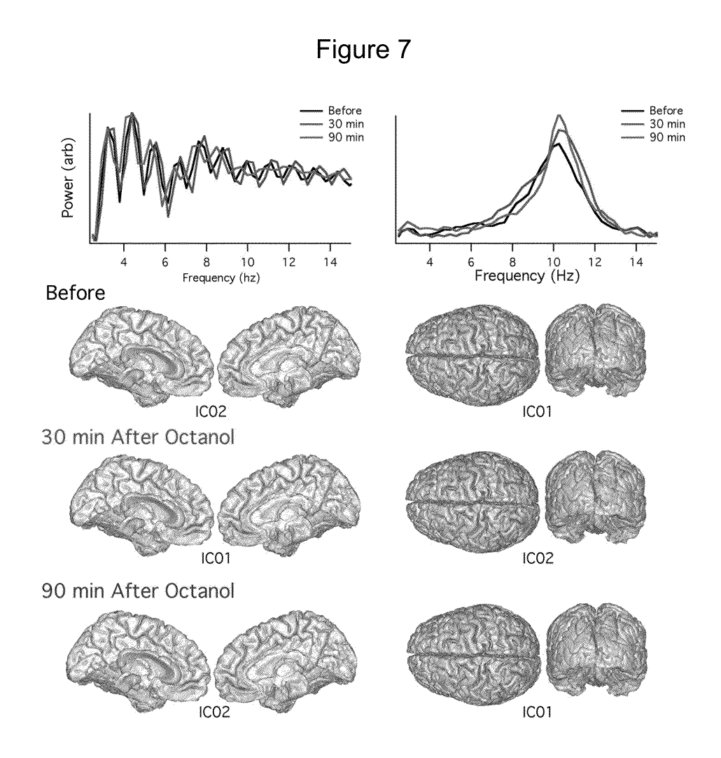

FIG. 7: No Change in Power. As shown in this example, low frequency peaks in some subjects, were not affected by octanol. Quite similar sources in low frequencies (left) and with alpha peaks (right) were very similar as were their localizations. This is very different from those low frequency peaks that were reduced or eliminated by octanol indicating a different underlying generating mechanism.

The above results indicate that 1-octanol increases normal, alpha range activity while reducing abnormal, delta range activity. It was reported as having no effect on general, self-reported measures of wellness. Reduction of these abnormal oscillations surgically through thalamic lesion has been shown to reduce symptoms in patients with TCD such as neuropathic pain and Parkinson's disease. Octanol is a promising candidate for a non-surgical approach to reduction of abnormal oscillations seen in TCD and relief of symptoms for some patients. Such low frequency activity has been shown to be due to hyperpolarization of thalamic neurons and low frequency oscillations supported by T-type calcium channels.

### Formulations of Octanol

### Example 2

### Preparation of Inclusion Compound of 1-octanol in β-cyclodextrin

### β-CD

2.27 g (0.002 mol) of β-CD (Sigma) was dissolved in 30 ml of water at room temperature under stirring. To the resulting solution was added 0.02 mol of 1-octanol (Sigma). After stirring at room temperature for about 24 hours, the solid particulates were collected by filtration, washed with water, and then lyophilized and/or let dry for one week, whereby 2.2 g of the target inclusion compound of 1-octanol in β-CD was obtained as a white powder.

The purity of the octanol inclusion molecules in the cyclodextrins may be determined by NMR analysis, as described below.

¹H-NMR analysis: ¹H-NMR spectrum (D₂O solution), as measured with tetramethylsilane (TMS) as an external standard, of a specific part of the structure spectrum of 1-octanol, in free form (A) and the specific part of the 1-octanol portion of the inventive compound; that is, inclusion compound of 1-octanol in β-CD (B). The difference in spectrum between A and B results from the inclusion by β-CD. The weight/weight ratio of 1-octanol toward octanol-cyclodextrin powder can also be assessed using the NMR analysis. The NMR analysis may also be used to determine the stability and resistance to decomposition for quality control purposes.

### Example 3

### Preparation of Inclusion Compound of 1-octanol in G1-β-cyclodextrin

### G1-β-CD

The inclusion compound can be prepared by adding 1-octanol to the solution of G1-β-CD and following the procedure described in the Example 2.

### Example 4

### Preparation of Inclusion Compound of 1-octanol in γ-cyclodextrin

### γ-CD

The titled inclusion compound can be prepared by adding 1-octanol to the solution of γ-CD and following the procedure described in Example 2. γ-CD is highly susceptible to saliva-amylase degradation, and thus it may be delivered using a lingual delivery method.

### Example 5

### Formulations of Inclusion Compound of 1-octanol in β-CD

Formulation with Vanilla-Bean Essential Oil

A 100-ml measuring flask was charged with 0.5 g of vanilla-bean essential oil (CO₂ supercritical extract) and 0.78 g (containing 0.1 g of octanol) of the inclusion compound of 1-octanol in β-CD (Example 2). The mixture was treated with 50% ethanol to give a total volume of 10 mL. Castor oil was then added to give the desired formulation.

### Chewing Gum

The inclusion compound of 1-octanol in β-CD prepared in Example 2 was mixed in a conventional manner in accordance with the below-described formulation, whereby 100 g of a chewing gum was prepared.

TABLE 1

| Components | Mixing ratio (wt. %) |
|---|---|
| Inclusion compound of 1-octanol in | 1 |
| β-CD (from Example 2) | |
| Vanilla flavor | 1.0 |
| Thick malt syrup | 13.0 |
| Gum-base resin | 20.0 |
| Powder sugar-sucralose 1-1 mix | Balance |

### Mouth-Meltable Pills

The mouth meltable pills may be prepared using the inclusion compound of 1-octanol in β-CD from Example 2 and mixing it using a conventional manner to form mouth-meltable pills.

### Homeopathic Pills

The inclusion compound of 1-octanol in β-CD from Example 2 was mixed in a conventional manner to form the pills.

### Hair Lotion

The inclusion compound of 1-octanol in G1-β-CD from Example 3 was mixed in a conventional manner in accordance with the formulation shown in Table 2, whereby 100 g of a hair lotion was prepared.

TABLE 2

| | Mixing ratio |
|---|---|
| Component | (wt. %) |
| Inclusion compound of CA in G1-β-CD | 2.0 |
| (from Example 2) | |
| β-Glycyrrhetic acid | 0.2 |
| Vanilla-bean essential oil-supercritical extraction | 0.5 |
| Polyoxyethylene hydrogenated castor oil | 5.0 |
| BHT (2,6-di-t-butyl-4-methylphenol) | 0.03 |
| Perfume | 0.05 |
| Purified water | Balance |

### Mouth Wash

The inclusion compound of 1-octanol in β-CD from Example 2 was mixed in a conventional manner in accordance with the formulation shown in Table 3, whereby 100 g of a mouth wash was prepared.

TABLE 3

| | Mixing ratio |
|---|---|
| Component | (wt. %) |
| Inclusion compound of CA in β-CD | 1.0 |
| (from Example 2) | |
| Ethanol | 12.5 |
| Sodium lauryl sulfate | 1.25 |
| Glycerin | 10.0 |
| Vanilla-bean essential oil-supercritical extraction | 0.5 |
| Saccharin | 0.001 |
| Coloring matter | 0.003 |
| Flavor | 0.05 |
| Purified water | Balance |

### Transmucosal

Lingual spray, sublingual pills, mouth dissolving strips and chewing gums may be prepared in conventional manner using the inclusion compound of 1-octanol in β-CD (Example 2).

### Slow or Time Released Octanol-CD Formulation

The slow released formulations may be prepared in conventional manner using the inclusion compound of 1-octanol in β-CD.

### Liquid Spray

The liquid spray formulations may be prepared in conventional manner using the inclusion compound of 1-octanol in β-CD or only octanol. A representative formulation is given below:

Deliver 1 mg octanol per 100 μl/spray-push

### Ingredients:

Castor oil: USP odorless tasteless (Amerisourcebergen). May later be replaced by Cremophor.
Syrup Toffee nut, coffee carb syrup (Starbucks)
Sucralose: pure powder
Vanilla: Pure Vanilla Extract (vanilla bean extract in water, alcohol (35%), and corn syrup). (Therapeutical grade CO₂ extraction of vanilla is available from specific suppliers).
EtOH: Absolute ethanol (lab grade)
Octanol
Water: distilled

Solution:
10% v/v Vanilla
15% v/v Toffenut syrup
2 mM Sucralose (2% v/v 100 mM)
5% v/v Castor Oil
60% v/v Ethanol
1% w/v octanol (=1.25% v/v)
complete to 100% with H₂O

Suggested Formulation for Pilot Production:
Sodium Benzoate 0.05%
Potassium Sorbate 0.05%
Sodium Citrate 0.25%
Sucralose ˜0.10%-0.5-2 mM Sucralose
Sucrose 10-15% w/v
Vegetable Glycerine 5.00%
Citric Acid, anhyd. 0.15%
Caramel color/flavor 2%
Flavors 0.x % Toffee Nut
Vanilla Oil 0.2-1% v/v
Castor Oil 5-10% (or Cremophor)
65% v/v Ethanol
1% w/v Octanol (=1.25% v/v)
Water x % to complete to 100%

### REFERENCES

1. Puil, E., Meiri, H., Yarom, Y., Journal of Neurophysiology 575 (1994).
2. Soltesz, I. et al., Journal of Physiology 441, 175 (1991).
3. Llinas, R., Muhlethaler, M., Journal of Physiology 404, 215 (1988).
4. Lampl, I., Yarom, Y., Neuroscience 78, 325 (1997).
5. Sinton, C M, Krosser, B I, Walton, K D, Llinas, R R, Eur J. Physiol. 414, 31 (1989).
6. Bushara, K. O., Goldstein, S. R., Grimes, G. J., Jr., Burstein, A. H., Hallett, M., Neurology 62, 122 (2004).
7. Shill, H. A., Bushara, K. O., Mari, Z., Reich, M., Hallett, M., Neurology 62, 2320 (2004).
8. Llinas, R. R., Ribary, U., Jeanmonod, D Kronberg, E., Mara, P. P., PNAS 96, 15222 (1999).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

The chemical names of compounds given in this application were generated using various commercially available chemical naming software tools including MDL's ISIS Draw Autonom Software tool, and were not verified. Particularly, in the event of inconsistency, the depicted structure governs.

1. (canceled)

2. A pharmaceutical composition or formulation comprising:
a) cyclodextrin; and
b) a C₈ alcohol,
wherein the pharmaceutical composition or formulation comprises an inclusion or caged or clathrate compound comprising cyclodextrin and a C₈ alcohol.

3. The pharmaceutical composition or formulation according to claim 2, wherein the inclusion compound is prepared by mixing cyclodextrin and a C₈ alcohol.

4. (canceled)

5. The pharmaceutical composition or formulation according to claim 2, wherein the inclusion compound is cyclodextrin encapsulated C₈ alcohol.

6. The pharmaceutical composition or formulation according to claim 2, wherein the cyclodextrin is α-cyclodextrin, β-cyclodextrin, γ-cyclodextrin, branched cyclodextrin, glucosyl cyclodextrin, diglucosyl cyclodextrin, maltosyl cyclodextrin, dimaltosyl cyclodextrin, or hydroxypropyl cyclodextrin.

7. (canceled)

8. The pharmaceutical composition or formulation according to claim 2, wherein the C₈ alcohol is 1-octanol.

9. The pharmaceutical composition or formulation according to claim 2, wherein the pharmaceutical composition or formulation further comprises natural vanilloid extracts, or oil based compounds.

10. (canceled)

11. The pharmaceutical composition or formulation according to claim 2, wherein the pharmaceutical composition or formulation further comprises oil based compounds selected from castor oil, hydrogenated castor oil, and vanilla-bean essential oil.

12. (canceled)

13. The pharmaceutical composition or formulation according to claim 2, wherein the pharmaceutical composition or formulation further comprises a carrier or adjuvant; and the carrier is a parenteral carrier, a oral carrier, a topical carrier, or a transmucosal carrier.

14. (canceled)

15. (canceled)

16. (canceled)

17. (canceled)

18. (canceled)

19. (canceled)

20. (canceled)

21. The pharmaceutical composition or formulation according to claim 2, wherein the pharmaceutical composition or formulation is incorporated in mouth meltable pills, mouth meltable strips, sublingual micro pills, gum, candy, ointment, gel, systemic lotion, systemic gel, massage cream, massage gel, mouth meltable pills, capsules, homeopathic pills, hair lotion, or mouth wash, or the pharmaceutical composition or formulation is delivered using a lingual spray, sublingual pill, a liquid spray, mouth dissolving strip or chewing gum.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. The pharmaceutical composition or formulation according to claim 2, wherein the pharmaceutical composition or formulation is used to treat a neurological disorder.

39. The pharmaceutical composition or formulation according to claim 38, wherein the neurological disorder is a thalamocortical dysrhythmia disorder.

40. The pharmaceutical composition or formulation according to claim 2, wherein the pharmaceutical composition or formulation is a low dosage formulation.

41. The low dosage pharmaceutical composition or formulation according to claim 40, wherein the dosage is in the range of 0.001 to 1 mg/kg or 0.01 to 0.1 mg/kg.

42. A method of treating a neurological disorder or condition in a mammal in need thereof, the method comprising administering to the mammal a therapeutically effective amount of the pharmaceutical composition according to claim 2.

43. The method according to claim 42, wherein the neurological disorder or condition is a thalamocortical dysrhythmia disorder, neurogenic pain, obsessive-compulsive disorder, depression, panic disorder, Parkinson's disease, schizophrenia, rigidity, dystonia, tinnitus, tremor, or epilepsy.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The method of claim 42, wherein the tremor is essential familial tremor (EFT).

58. The method of claim 42, wherein the therapeutically effective amount administered is from about 0.001 mg/kg body weight to about 20 mg/kg body weight, about 0.1 mg/kg body weight to about 10 mg/kg body weight, or 0.3 mg/kg body weight to about 3.0 mg/kg body weight of the mammal.

59. (canceled)

60. (canceled)

61. (canceled)

62. (canceled)

63. (canceled)

64. A method of improving alertness and attentiveness, improving focal concentration capability, enhancing cognition, or promoting nootropic activity in the healthy humans; or a method to aid meditative states in the healthy humans, the method comprising administering to the mammal a therapeutically effective amount of the pharmaceutical composition according to claim 2.

65. (canceled)

66. (canceled)

67. (canceled)

68. (canceled)

69. (canceled)

70. (canceled)

71. (canceled)

72. (canceled)

73. A low dosage composition or formulation comprising 1-octanol, wherein the therapeutically effective amount of 1-octanol administered is from 0.01 to 1 mg/kg or 0.01 to 0.1 mg/kg.

74. (canceled)

75. The composition or formulation according to claim 73, wherein composition or formulation is incorporated in mouth meltable pills, mouth meltable strips, sublingual micro pills, gum, candy, ointment, gel, systemic lotion, systemic gel, massage cream, massage gel, mouth meltable pills, capsules, homeopathic pills, hair lotion, or mouth wash.

76. The composition or formulation according to claim 73, wherein the composition or formulation is delivered using a lingual spray, mouth meltable pill, sublingual pill, mouth dissolving strip or chewing gum.

77. (canceled)

78. (canceled)

79. (canceled)

80. A method of preparation of an inclusion compound of a C₈ alcohol and cyclodextrin.

81. (canceled)

82. (canceled)

83. (canceled)
